(19) United States
(12) Patent Application Publication
(10) Pub. No.: US 2020/0352791 A1
(43) Pub. Date: Nov. 12, 2020

(54) PATIENT SUPPORT PLATFORM TO DRAIN LIQUID WASTE

(71) Applicant: Lex Nakashima, Arleta, CA (US)

(72) Inventor: Lex Nakashima, Arleta, CA (US)

(21) Appl. No.: 16/407,513

(22) Filed: May 9, 2019

(51) Int. Cl. A61F 13/15

(52) U.S. Cl. CPC A61F 13/15; A61F 2013/15146; A61F 2013/5113; A61F 2013/15186; A61F 2013/15495; A61F 2013/15154

(57) ABSTRACT

A riser provides a support platform for a patient and includes a resilient pad covered by mesh material for supporting a patient. The mesh material and resilient pad are porous and allow liquid waste from the patient to pass through to an absorbent pad, keeping the patient dry and separated from the liquid waste.

### BACKGROUND

One current method to deal with the liquid waste (urine) of a moribund patient is to use an absorbent pad. The problem is that the patient remains in contact with the damp, soiled pad. It would be desirable to have a device that simply and effectively isolates the patient from the liquids, which will keep the patient dry and comfortable. Furthermore, it would also be desirable to have a device that stabilizes and secures absorbent pads from patient activity. Still further, it would be desirable to have a device that helps create a sanitary, comfortable surface for the patient to lie on. Therefore, there currently exists a need in the industry for a device and associated method that allows liquid waste from a moribund patient to both drain away and keep the patient isolated from that liquid.

Currently all of the methods for managing waste liquids of patients who have restricted movement are limited to absorbent pads (“pee-pads”) or wire platforms through which the liquid drains through. The solutions that attempt to absorb the liquids, via pads or absorbent materials fail to meet the needs of the industry because the patient is still in contact with the damp surface of the absorbent pad (unsanitary, uncomfortable) and the pad itself gets bunched up due to even the slightest movement by the patient. These absorbent pads also require the patient to be physically handled and moved when the pads need to be replaced. Also, unless the patient is constantly monitored, the patient remains in contact with the damp, soiled surface of the pad until it is noticed that the pad has become soiled, which can be an uncomfortable amount of time.

One solution, which uses a raised metal grating or wire mesh to allow liquid waste to drain through, fails because the surface is both uncomfortable and, due to the open nature of the grate, leads to patient body heat loss.

It would be desirable to have a device that simply and effectively isolates patient from the liquids, which will keep the patient dry and comfortable. Furthermore, it would also be desirable to have a device that stabilizes and secures absorbent pads from patient activity. Still further, it would be desirable to have a device helps create a sanitary, comfortable surface for the patient to lie on. Therefore, there currently exists a need in the industry for a device and associated method that allows liquid waste from a moribund patient to both drain away and keep the patient isolated from that liquid.

### SUMMARY OF THE INVENTION

The present invention relates to a medical device and a method associated with the device. With respect to the device, a porous riser acts as a platform and is made from an elastic, open-celled material, which allows fluids to drain through. Rather than simply absorb any type of liquid, such as waste liquids (urine), the device acts as a riser to separate a patient and liquids. The device can be used in the care of human patients in a hospital or other care facility, elderly pets with incontinence, pets in travel kennels for extended periods, or veterinary hospitals for recovering pets.

In one embodiment, the riser includes a resilient pad made of reticulated (open celled) polyurethane foam, compressed fibers, rubberized hair, rubberized fiber, or other porous material, and is covered in a nylon, polyester or polypropylene mesh material with hole size not greater than 0.3 inch (9 holes per square inch). The open mesh covers the porous resilient pad for comfort, with this platform on top of a drain pan or absorbent material. The dimensions of the riser will vary with respect to the area the riser will be located and the weight of the patient (thicker for heavier patients).

With respect to the associated method, the riser is constructed of a material that is porous enough to allow liquids to quickly and freely flow though, while remaining resilient enough to maintain separation between patient and liquid. Ultimately, at the conclusion of these steps the patient will stay considerably dryer and more comfortable than without.

As used herein, the word “patient” refers to any animal, human or non-human (i.e., dogs, cats, other).

### BRIEF DESCRIPTION OF THE DRAWINGS

FIG. 1 is a perspective view of a riser acting as a conduit to separate a patient from liquid waste.

FIG. 2 is a top view of the riser.

FIG. 3 is a bottom view of the porous riser depicting support webbing.

FIG. 4 is a front view of the carry handles used to move a patient laying on top of the riser.

FIG. 5 is a back view of the carry handles used to move a patient laying on top of the riser.

FIGS. 6A and 6B are left and right side views, respectively, of the riser depicting nylon webbing along the sides.

FIG. 7 is a partial cross-sectional view of the riser depicting liquid waste passing through the mesh and resilient pad and into the absorbent pad.

FIG. 8 is a partial exploded view of the riser, bottom-side up, depicting the insertion area for an absorbent pad.

FIG. 9 is a perspective elevation view depicting use of the handles to transport the patient on top of the riser.

### DETAILED DESCRIPTION OF THE PREFERRED EMBODIMENTS

As shown in the drawings, a riser 10 is used to drain liquid waste from a patient to a receptical or absorbent pad. Importantly, the riser is a platform that supports a patient and that provides spacing between the patient and the receptical or absorbent pad so that the patient does not remain in contact with the liquid waste.

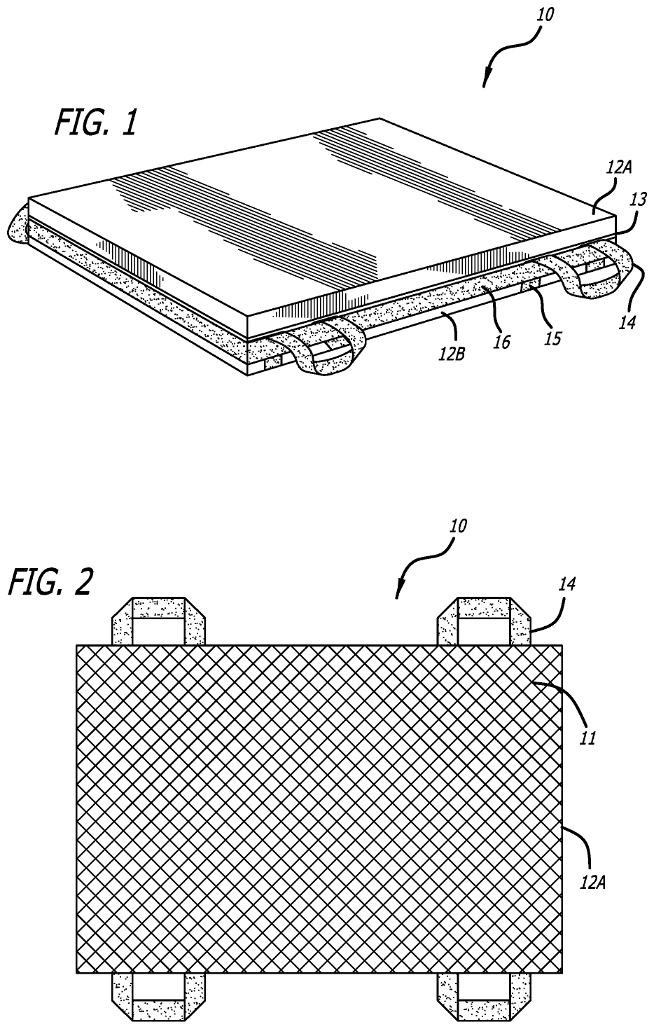

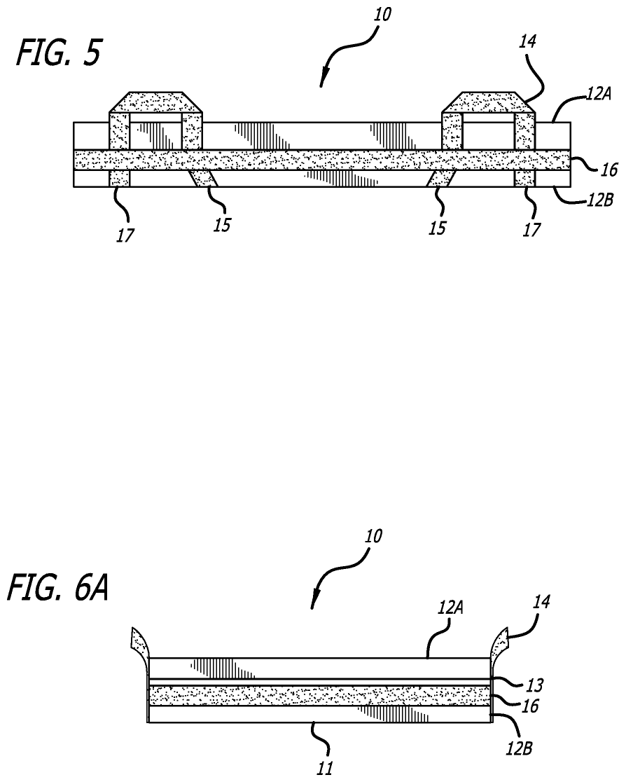

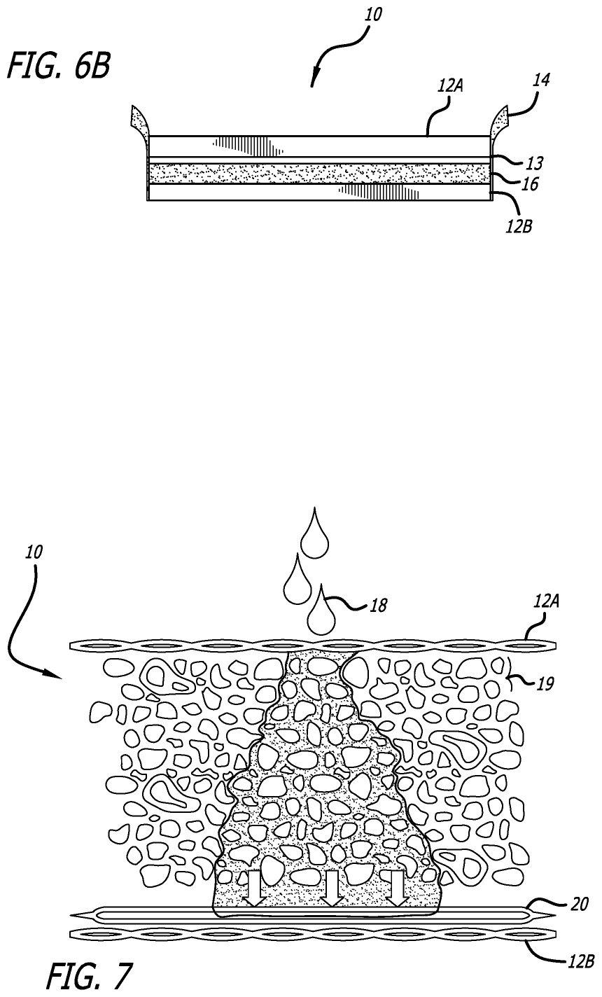

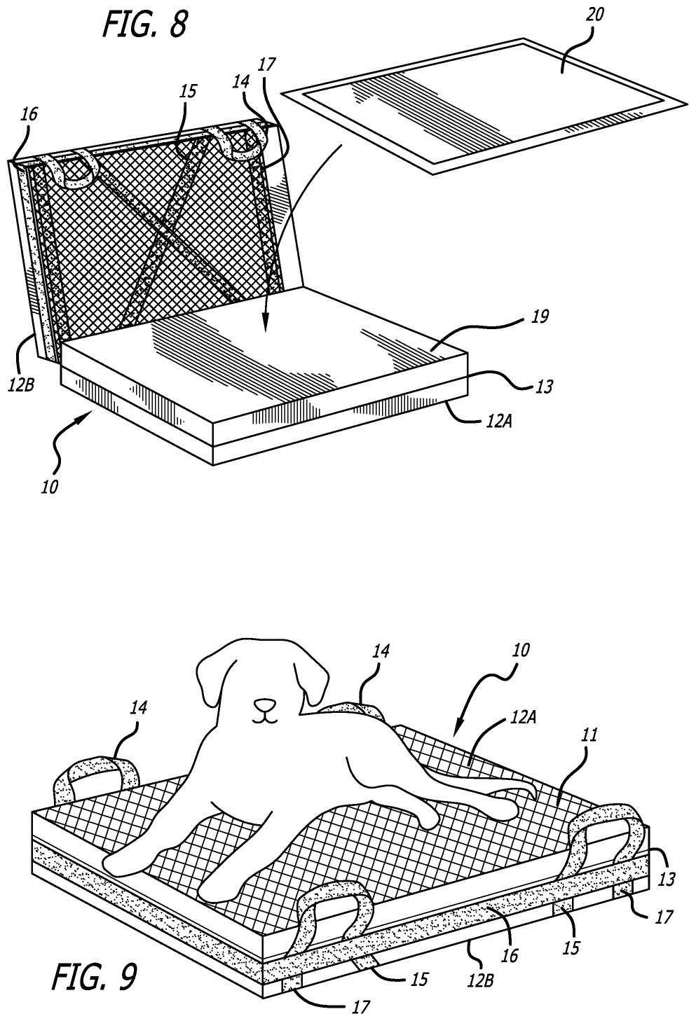

In one embodiment, as shown in FIGS. 1-9, the riser 10 acts as a conduit to keep separate a patient from liquid waste. The riser 10 is covered in a mesh cover 11, which surrounds the riser and has top portion 12A and a bottom portion 12B. The mesh cover 11 envelops a resilient pad 19 made from a porous or open material that is elastic and that allows liquids to drain through quickly. The mesh cover 11 is made from an open mesh material. The porous riser is inserted into the mesh cover which creates a strong, comfortable surface that the patient lies upon, and through which liquid waste drains through. In one embodiment, the mesh cover 11 has holes that do not exceed 0.3 inch in diameter. In another embodiment, the mesh cover 11 has holes that do not exceed 0.5 inch in diameter. In another embodiment, the holes in the mesh cover 11 do not exceed nine uniformly spaced holes per square inch of material. In another embodiment, the holes in the mesh cover 11 do not exceed sixteen spaced holes per square inch of material.

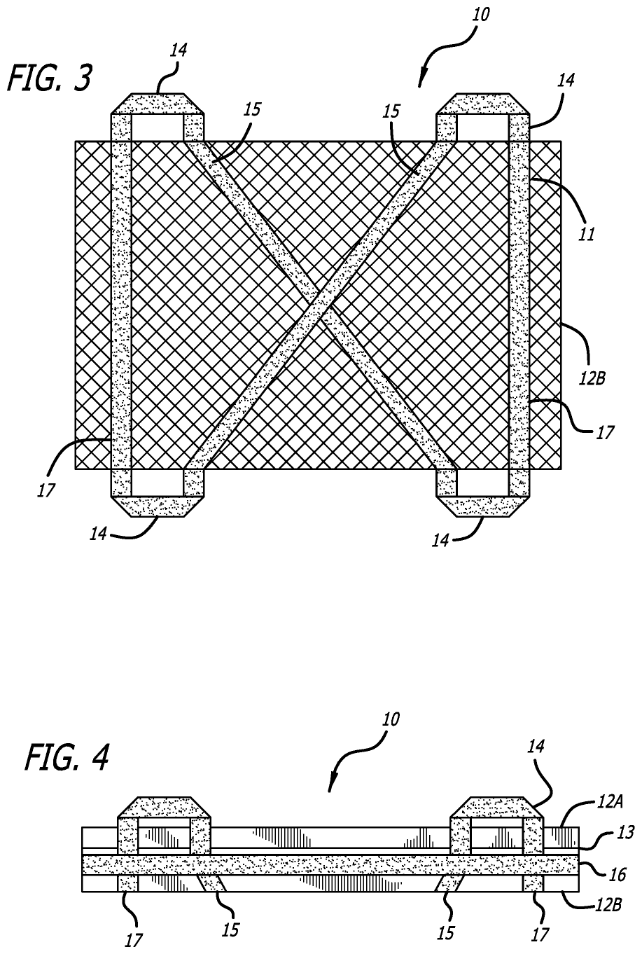

In another embodiment, as shown in FIGS. 1-6B, nylon or other durable webbing straps 15, 16 and 17 are attached to the mesh cover 11 for additional support. The X-shaped webbing straps 15 are attached to the mesh cover bottom 128 diagonally across the mesh cover (in an “X” pattern as shown in FIGS. 3 and 4). These webbing straps 15, 16 and 17 reinforce the structure of the riser 10. Side webbing straps 16 extend around the sides of the riser 10 and are attached to the sides of the mesh cover 11. The vertical webbing straps 17 extend along the same bottom surface 12B of the mesh cover 11 as the X-shaped webbing straps 15. All of the webbing straps 15, 16 and 17 are attached to the mesh cover 11 by means of stitching, adhesive, VELCRO® attachment, or other similar means. In one embodiment, the attachment of the webbing straps 15, 16 and 17 is continuous along all contact surfaces between the straps and the mesh cover. The shape and position of the webbing straps 15, 16 and 17 is critical to the structure of the riser 10 to maintain the shape of the riser when a patient is on the riser or being transported on the riser. Further, the webbing straps 15, 16 and 17 are critical to keep the sides of the riser 10 from collapsing under load and to prevent shifting of the mesh cover 11 relative to the resilient pad 19. Thus, the webbing straps 15, 16 and 17 add significant support structure to the riser 10. Handles 14 can be attached to the straps on both sides (two per side) to allow carrying or moving the patient. The handles 14 can be made from the same materials as the webbing straps 15, 16 and 17, such as nylon. Additionally, supplemental straps (not shown) can be used to secure the patient during transport.

As can be seen in FIG. 3, in one embodiment, the handles 14 and the webbing straps 15 and 17 are formed from a single, continuous strap. In another embodiment, the handles 14 and the X-shaped webbing straps 15 are formed from a single continuous strap. In another embodiment, the handles 14 and the vertical webbing straps 17 are formed from a single continuous strap. In another embodiment, the handles 14 are attached to the mesh cover 11 and to the X-shaped webbing straps 15 and the vertical webbing straps 17 by stitching, adhesives, VELCRO®, or similar means. Importantly, the stress placed on the handles when moving the riser 10 with a patient is evenly distributed along the X-shaped webbing straps 15 and the vertical webbing straps 17, thereby providing a firm structure or platform that does not shift or distort under load.

In one embodiment, as shown in FIGS. 4 and 9, the handles 14 pass under the side webbing straps 16. Since the side webbing straps 16 are securely attached to the sides of the mesh cover 11, they add reinforcement to the attachment of the handles 14 which come under load when moving a patient.

In another embodiment, as shown in FIGS. 1-9, the riser 10 includes a resilient pad 19 formed of an open-celled or reticulated foam; a mesh cover 11 encapsulating the pad; a zipper 13 (or VELCRO® or snaps) or other closure devices to allow an absorbent pad 20 to be inserted and contained under the resilient pad 19; reinforcing webbing or straps 15, 16, 17; and handles 14. These components are assembled by encasing the resilient pad 19 inside the mesh cover 11, while the absorbent pad 20 is inserted inside the mesh cover 20 and under the resilient pad. It should further be noted that the resilient pad material must be able to conduct liquids quickly from top to bottom using only gravity and the material should be such that it can be easily cleaned and sterilized, and dry quickly for reuse. The resilient pad 19 is configured to easily shed waste material with a simple flow of water from a hose or by washing in a sterile environment. Thus, the resilient pad 19 has pore sizes having 70% to 99% open porosity. The thickness of the resilient pad 19 will vary depending upon the weight of the patient. It is possible that the patient can weigh as little as a few pounds up to 250 pounds or more. Thus, the resilient pad 19 has a thickness from 0.25 inch up to 12.0 inches, depending upon the weight of the patient. A resilient pad 19 having a thickness of four inches would be capable of supporting a patient weighing up to 180 pounds, while a six inch resilient pad will support a patient weighing up to 300 pounds. The length and width of the resilient pad 19 also is variable depending on the size of the patient. In one embodiment the resilient pad 19 ranges from one foot wide to five feet wide, and from two feet long to 8 feet long.

In one embodiment, the resilient pad 19 is made of a reticulated (open celled) polyurethane foam, compressed fibers, vitreous carbon foam, rubberized hair, rubberized fiber or other porous material, which is available as DRYFAST FOAM® from Foam Factory, Inc., Michigan, or REFRACTORY OPEN-CELL FOAM® from Ultramet, Calif., or RETICULATED (Filter) FOAM® from All Foam Products Co., Inc., Ohio. The mesh cover 11 is made of nylon, polyester, or polypropylene mesh material with a hole size not greater than 0.3 inch or nine holes per square inch, and is available as XB1130 BLACK POLYETHYLENE MESH® from Industrial Netting, Minnesota, or 3MM BLACK AIRMESH N5® from Eastex Products, Inc., Massachusetts, or NH43® from Apex Mills, North Carolina.

In one embodiment, the resilient pad 19 is comprised of two layers, each layer being four inches thick of 10 ppi reticulated foam. In another embodiment, the resilient pad 19 is comprised of two layers, a first layer being two inches thick of 10 ppi reticulated foam on top of a second layer being two inches thick of 20 ppi reticulated foam. In another embodiment, the resilient pad 19 is comprised of one layer being two inches thick of 10 ppi reticulated foam. In another embodiment, the resilient pad 19 is comprised of two layers, the first layer being one inch thick of 10 ppi reticulated foam on top of a second layer being one inch thick of 20 ppi reticulated foam. In another embodiment, the resilient pad 19 is comprised of two layers, the first layer being no greater than one inch thick of 20 ppi reticulated foam on top of a second layer being one inch thick of 10 ppi reticulated foam.

One method of use associated with the disclosed device comprises the following steps: (1) encasing the resilient pad 19 in the mesh cover 11; (2) sealing the mesh cover 11 closed using a zipper or VELCRO® 13 (or other form of closure device); (3) insert the absorbent pad 20 inside the mesh cover 11 and below the bottom surface of the resilient pad; (4) position patient on the top surface 12A of the mesh cover 11; (5) maintain spacing between the patient and the absorbent pad; and (6) allow liquid waste 18 from the patient to travel through the mesh cover 11 and the resilient pad 19 into the absorbent pad 20. When liquid waste is created, the liquid will flow freely through the riser 10 and be caught/absorbed by the absorbent pad (sometimes known as a “pee-pad”). Thus, the patient remains dry, and is kept physically away from the liquid waste. It should further be noted that this invention is intended for use with patients where liquids (water, waste, etc.) need to be isolated from the body because the patient is either incontinent, restricted, unconscious, sleeping, or moribund. The webbing strap reinforcement 15 and handles 14 will allow caregivers to move the patient to another location; lift the patient to change out the soiled pad; and re-position the patient on the riser 10. With multiples of the riser 10, the patient can be transferred to a clean riser while the soiled riser is cleaned and sterilized. Because the patient is constantly separated from the liquid waste, this cleaning may be done as required.

The resilient pads are cleaned with a combination of water and disinfectant (eg, bleach), using a simple garden hose or sink. The cleaned pads can then either be placed vertically to drain and air-dry, or use a rack-and-fan to dry them more rapidly.

The disclosed device is unique when compared with other known devices and solutions because it: (1) keeps the patient physically apart from liquid waste; (2) provides the patient with an elastic, comfortable surface on which to lay; (3) allows for the patient to remain dry despite any lag between soiling and response of caregivers; (4) is easy to clean and dry; and (5) allows for air to contact points of the patient's body, while still preserving patient body heat. This can prevent bed sores from lack of air circulation on the skin, and possibly prevent infant suffocation in a crib situation. Similarly, the associated method is unique in that it: (1) does not seek to merely contain liquids, it conducts the liquids away from the patient; (2) allows for air to contact points of the patient's body, while still preserving patient body heat; and (3) is easy to use, re-use, and maintain. Similarly, the disclosed method is unique when compared with other known processes and solutions in that it: (1) creates a stable, dry comfortable surface for the patient; (2) minimizes the necessity to move the patient; and (3) makes care for these patients simpler and more sanitary.

The process associated with the invention is likewise unique. More specifically, the disclosed process is unique because it: (1) uses separation and drainage for the comfort of the patient, rather than merely containing the liquid waste; (2) by placement of the absorbent pad beneath the thick, porous resilient pad, the absorbent pad is securely stabilized; and (3) due to the porous nature of the material of the resilient pad, the pad dries quickly, which facilitates both patient comfort and quick and easy cleaning.

I claim:

1. A medical apparatus for use by a patient, comprising:
a porous riser including a resilient pad having a length, a width and a thickness for allowing fluids to pass therethrough;
a mesh cover for encapsulating the resilient pad;
a collection device to collect the fluids passing through the resilient pad; and
wherein a density of the resilient pad is sufficient to maintain a spacing between the porous riser and the collection device when a patient is positioned on the porous riser and porous enough to allow liquids to pass freely therethrough.

2. The medical apparatus of claim 1, wherein:
the resilient pad is formed from an elastic, open-celled material.

3. The medical apparatus of claim 2, wherein the elastic, open-celled material is taken from the group of materials including polyurethane foam and compressed fibers.

4. The medical apparatus of claim 3, wherein the resilient pad comprises pore sizes having 70% to 99% open porosity.

5. The medical apparatus of claim 2, wherein the resilient pad comprises a vitreous carbon foam.

6. The medical apparatus of claim 1, wherein the thickness of the resilient pad is in the range from 0.25 inch to 12.0 inch.

7. The medical apparatus of claim 1, wherein the mesh cover is formed from the group of mesh materials including nylon, polyester and polypropylene.

8. The medical apparatus of claim 7, wherein the mesh materials have a hole size not greater than 0.5 inch.

9. The medical apparatus of claim 7, wherein the mesh materials have no greater than sixteen spaced holes per square inch.

10. The medical apparatus of claim 1, wherein the collection device is a bed pan.

11. The medical apparatus of claim 1, wherein the collection device is an absorbent material.

12. The medical apparatus of claim 11, wherein the absorbent material is removably inserted into the mesh cover.

13. A medical apparatus for use by a patient, comprising:
a porous riser including a resilient pad having a length, a width and a thickness allowing fluids to pass therethrough;
a mesh cover removably attached to the resilient pad;
a collection device to collect the fluids passing through the resilient pad; and
wherein a density of the resilient pad is sufficient to maintain a spacing between the porous riser and the collection device when a patient is positioned on the porous riser.

14. The medical apparatus of claim 13, wherein:
the resilient pad is formed from an elastic, open-celled material.

15. The medical apparatus of claim 14, wherein the elastic, open-celled material is taken from the group of materials including polyurethane foam and compressed fibers.

16. The medical apparatus of claim 15, wherein the resilient pad comprises pore sizes having 70% to 99% open porosity.

17. The medical apparatus of claim 13, wherein the mesh cover is formed from the group of mesh materials including nylon, polyester and polypropylene.

18. The medical apparatus of claim 17, wherein the mesh materials have no greater than sixteen spaced holes per square inch.

19. The medical apparatus of claim 13, wherein the collection device is an absorbent material.

20. The medical apparatus of claim 19, wherein the absorbent material is removably inserted into the mesh cover.

21. A medical apparatus for use by a patient, comprising:
a porous riser provides a support platform for a patient and includes a resilient pad having a length, a width and a thickness allowing fluids to pass therethrough;
a mesh cover removably attached to the resilient pad;
wherein the porous riser includes reinforcing straps along a top surface, a bottom surface, and side surfaces;
a collection device to collect the fluid passing through the resilient pad; and
wherein a density of the resilient pad is sufficient to maintain a spacing between the resilient pad and the collection device when a patient is positioned on the porous riser.
